SEQUENCE LISTING (10 sequences)

SEQ ID NO: 1 - DNA, 20 bp, Artificial Sequence
  GPR119 forward primer
tgcagctgcctctgtcctca

SEQ ID NO: 2 - DNA, 20 bp, Artificial Sequence
  GPR119 reverse primer
gcacaggagagggtcagcac

SEQ ID NO: 3 - DNA, 20 bp, Artificial Sequence
  beta-actin forward primer
ccacagctgagagggaaatc

SEQ ID NO: 4 - DNA, 20 bp, Artificial Sequence
  beta-actin reverse primer
aaggaaggctggaaaagagc

SEQ ID NO: 5 - DNA, 21 bp, Artificial Sequence
  SCD1 forward primer
gctgctcggatcactagtgaa

SEQ ID NO: 6 - DNA, 21 bp, Artificial Sequence
  SCD1 reverse primer
ttctgctatcagtctgtccag

SEQ ID NO: 7 - DNA, 20 bp, Artificial Sequence
  FAS forward primer
agtacacacccaaggccaag

SEQ ID NO: 8 - DNA, 20 bp, Artificial Sequence
  FAS reverse primer
ggatactttcccgtcgcata

SEQ ID NO: 9 - DNA, 20 bp, Artificial Sequence
  beta-actin forward primer(in Table 2)
gatgagattggcatggcttt

SEQ ID NO: 10 - DNA, 20 bp, Artificial Sequence
  beta-actin reverse primer(in Table 2)
gtcaccttcaccgttccagt

(19) United States
(12) Patent Application Publication
(10) Pub. No.: US 2017/0049773 A1
(43) Pub. Date: Feb. 23, 2017

(54) PHARMACEUTICAL COMPOSITION CONTAINING GPR119 LIGAND AS ACTIVE INGREDIENT FOR PREVENTING OR TREATING NON-ALCOHOLIC FATTY LIVER DISEASE

(71) Applicants: DONGGUK UNIVERSITY INDUSTRY-ACADEMIC COOPERATION FOUNDATION, Seoul (KR); SNU R&DB FOUNDATION, Seoul (KR)

(72) Inventors: Keon Wook KANG, Seoul (KR); Kyeong LEE, Goyang-si, Gyeonggi-do (KR); Jin Won YANG, Seoul (KR)

(73) Assignees: DONGGUK UNIVERSITY INDUSTRY-ACADEMIC COOPERATION FOUNDATION, Seoul (KR); SNU R&DB FOUNDATION, Seoul (KR)

(21) Appl. No.: 15/302,228

(22) PCT Filed: Jan. 23, 2015
(86) PCT No.: PCT/KR2015/000766

§ 371 Date: Oct. 6, 2016

(30) Foreign Application Priority Data
Jan. 23, 2014 (KR) 10-2014-0008368
Jan. 22, 2015 (KR) 10-2015-0010771

(51) Int. Cl. A61K 31/506; A61K 31/4545

(52) U.S. Cl. CPC A61K 31/506; A61K 31/4545

(57) ABSTRACT

The present invention relates to a pharmaceutical composition containing a G protein coupled receptor 119 (GPR119) ligand as an active ingredient for preventing or treating non-alcoholic fatty liver disease. More particularly, it was confirmed that the GPR119 ligand, which has been developed as only an anti-diabetic drug, exhibits superior effects on the treatment of non-alcoholic fatty liver and the signal pathways in hepatocytes therefor differ from the signal pathways in the small intestine and the pancreas exhibiting anti-diabetic effects, whereby the GPR119 ligand can be useful to treat non-alcoholic fatty liver.

### TECHNICAL FIELD

The present invention relates to a pharmaceutical composition containing a G protein coupled receptor 119 (GPR119) ligand as an effective ingredient for preventing or treating non-alcoholic fatty liver disease.

### BACKGROUND ART

Fatty liver is characterized by abnormal fat accumulation in hepatocytes and medically refers to a pathological state wherein triglycerides make up 5% or more of a total weight of the liver. In general, fatty liver is characterized by alcoholic fatty liver disease (ALD), which is caused by continuous and excessive drinking, and non-alcoholic fatty liver disease, which exhibits hepatic tissue manifestation similar to the alcoholic fatty liver with little alcohol intake history.

Alcoholic fatty liver, as a frequently occurring disease in South Korea, develops due to facilitated fat synthesis in the liver by alcohol drinking and abnormal energy metabolism. Such alcoholic fatty liver can develop into hepatitis or cirrhosis depending upon alcohol intake.

Non-alcoholic fatty liver may occur due to causes such as obesity, diabetes, hyperlipidemia, and drugs, regardless of drinking. Non-alcoholic fatty liver refers to a wide range of diseases including simple fatty liver (steatosis) which is not accompanied by inflammation, non-alcoholic steatohepatitis (NASH) which exhibits hepatocellular inflammation, advanced fibrosis, and cirrhosis, depending upon the stage of development.

In modern society, 20 to 30% of the adult population of advanced countries suffers from non-alcoholic fatty liver disease (NAFLD) due to the increase of adult diseases according to high-fat and high-calorie food intake. 2 to 3% thereof develop non-alcoholic steatohepatitis (NASH). In particular, the patients histologically exhibit steatohepatitis manifestation accompanied by fibrosis and inflammation and, accordingly, are at high risk of developing cirrhosis, liver failure, and liver cancer.

Although it has been reported that steatohepatitis is closely related to obesity, insulin resistance, type II diabetes, etc., active research into definite pathogenesis thereof is actively underway. Due to the “two-hit” hypothesis recently suggested, abnormal insulin resistance related to and occurring with obesity, which is initiated by fats accumulated in the hepatocytes, and diabetes, etc. is considered the main cause of steatohepatitis. In addition, increased free fatty acids (FFAs) in hepatocytes, lipotoxicity due to cholesterol, and increased inflammatory cytokines and receptors thereof are reported to perform important functions in progressing from fatty liver to steatohepatitis.

According to an announcement by Statistics Korea in 2009, South Korea has the highest mortality rate due to liver cancer among OECD nations, and thus, social/economic losses are great. In addition, the domestic market for drugs for liver diseases reached 75.4 billion KRW in 2010, which is 4% higher than the 72.5 billion KRW in 2009. The drug market for liver diseases is anticipated to continue to increase henceforth. Further, the overseas market for hepatic protectors was 620 million KRW in 2003 and was anticipated to increase by about 10 to 35% in five years. The scale of the domestic health functional food market increased from 1.2 trillion KRW in 2003 to 1.5 trillion in 2004 and was anticipated to reach 2 trillion KRW in 2010. According to a report by BCC Research, a global consulting company, the market for drugs for treating non-alcoholic fatty liver in 2007 was estimated to be eight billion USD.

At present, drugs used by alcoholic or non-alcoholic fatty liver patients are broadly classified into two types as follows: 1) drugs for treating or improving fatty liver by curing risk factors, such as diet pills (orlistat), drugs for treating insulin resistance (metformin, pioglitazone, and rosiglitazone), and drugs for treating hyperlipidemia (clofibrate, gemfibrozil, bezafibrate, atorvastatin, simvastatin), and 2) drugs for recovering previously damaged hepatocyte and liver function, such as drugs for protecting hepatocytes (ursodeoxycholic acid and taurine), antioxidants (vitamin E), and nutritional supporters (lectin, betaine, and N-acetylcysteine), regardless of curing of risk factors of fatty liver. However, such conventionally used drugs are not fundamental therapeutic agents and are used as agents for improving symptoms, and when efficacy is considered, they cannot provide target effects. Until now, there has been little development in therapeutic agents for pharmacologically treating fatty liver, and thus, great ripple effects are anticipated when suitable drugs are developed.

A key concept in modern drug development is a target protein. The target protein refers to a protein whose function is changed due to the action of therapeutic agents and which can thus affect disease development. A fundamental first step in developing new drugs is to develop compounds that can selectively act on target proteins. However, since research on various candidate materials for treating non-alcoholic fatty liver has been conducted based on animal or cellular experiments, precise action points of action thereof have not been determined. In addition, in the case in which natural substances are utilized, compounds therein are unclear in many cases. Therefore, there are limitations in developing new drugs.

The human GPR119 gene is located on the X chromosome and is composed of a single exon. The composition of human GPR119 differs from those in mice or rats, but a protein expressed from the human GPR119 gene is very similar to that of a mouse. A G protein having seven transmembrane domains like other G protein-coupled receptors and interactively working in the cellular environment has not been clarified yet.

A GPR119 receptor was reported to be mainly present in beta cells of the pancreas and K cells and L cells, as secretory cells, in the small intestine. It is known that, due to activation of GPR119 in the pancreas, adenylate cyclase in cells increases the level of cAMP, as a second messenger, and thus, insulin secretion is increased upon external glucose stimulation. Such insulin secretion facilitation depends upon glucose stimulation. Accordingly, the GPR119 receptor is advantageous in that it does not exhibit hypoglycemia-inducing effects observed in conventional antidiabetics. GPR119 activation in the small intestine is reported to facilitate the secretion of GLP-1, GLP-2, and peptide YY in L-cells and the secretion of insulinotropic peptide (GIP) in K-cells. All of the aforementioned GPR119 functions are related to signals that facilitate hypoglycemic action, and thus, anti-diabetic effects can be anticipated. In practice, administration of a GPR119 ligand was reported to lead to effectively improved tolerance in in vivo mouse models subjected to an insulin tolerance test (ITT) and a glucose tolerance test (GTT).

As endogenous GRP119 ligands, there are human lipid-like substances such as N-acylethanolamines (NAEs), e.g., OEA, PEA, and LEA. These were reported to have affinity to, other than GPR119, receptors such as PPAR alpha and TRPV1. Large pharmaceutical companies such as Arena Pharmaceuticals and GlaxoSmithKline (GSK) manufacture synthetic GPR119 ligands as illustrated in the following drawing. Such synthesized GPR119 ligands are anticipated as next-generation drugs for most diabetes treatments. In particular, MBX2982 and GSK1292263, as the most actively studied substances, are in phase II clinical trials.

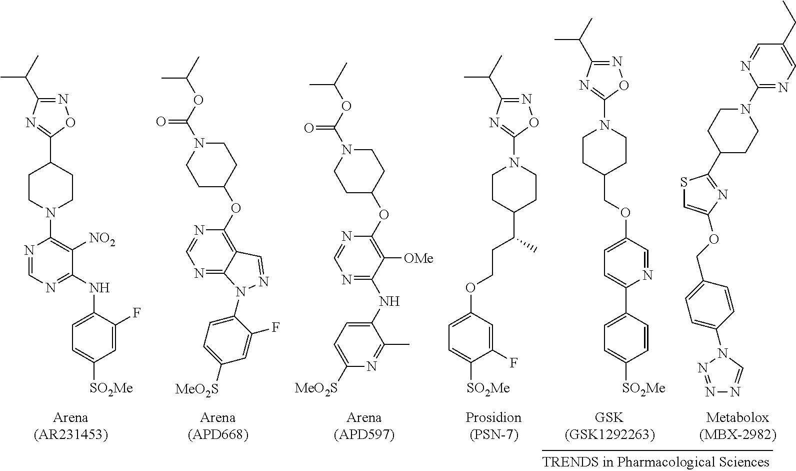 [SMILES: CC(C)c1noc(C2CCN(c3ncnc(Nc4ccc(S(C)(=O)=O)cc4F)c3[N+](=O)[O-])CC2)n1 ; CC(C)OC(=O)N1CCC(Oc2ncnc3c2cnn3-c2ccc(S(C)(=O)=O)cc2F)CC1 ; COc1c(Nc2ccc(S(C)(=O)=O)nc2C)ncnc1OC1CCN(C(=O)OC(C)C)CC1 ; CC(C)c1noc(N2CCC([C@H](C)CCOc3ccc(S(C)(=O)=O)c(F)c3)CC2)n1 ; CC(C)c1noc(N2CCC(COc3ccc(-c4ccc(S(C)(=O)=O)cc4)nc3)CC2)n1 ; CCc1cnc(N2CCC(c3nc(OCc4ccc(-n5cnnn5)cc4)cs3)CC2)nc1]

In previous studies, expression of GPR119 was hardly observed in the liver, and thus, efficacy of the GPR119 ligand on fatty liver was not evaluated (Odori S et al., Metabolism, 2013). In addition, due to the fact that free fatty acid and triglyceride levels in the blood of GPR119 gene-deficient mice are not higher than those of normal mice, the effects of the GPR119 receptor ligand on non-alcoholic fatty liver were not evaluated (Lan et al., 2009, J. Endocrinol.).

### DISCLOSURE

### Technical Problem

Therefore, the present invention has been made in view of the above problems. The present invention confirms that a ligand acting on a GPR119 receptor exhibits therapeutic effects on non-alcoholic fatty liver, and thus, it is an object of the present invention to provide a GPR119 ligand suitable for preventing or treating non-alcoholic fatty liver disease.

It will be understood that technical problems of the present invention are not limited to the aforementioned problems and other technical problems not referred to herein will be clearly understood by those skilled in the art from disclosures below.

### Technical Solution

One aspect of the present invention provides a pharmaceutical composition containing a G protein coupled receptor 119 (GPR119) ligand as an effective ingredient for preventing or treating non-alcoholic fatty liver disease.

The GPR119 ligand may be 4-((4-(1H-tetrazol-1-yl)phenoxy)methyl)-2-(1-(5-ethylpyrimidin-2-yl)piperidin-4-yl)thiazole (MB X2982) or 3 -isopropyl-5 -(4-(((6-(4-(methylsulfonyl)phenyl)pyridin-3-yl)oxy)methyl)piperidin-1-yl)-1,2,4-oxadiazole (GSK1292263).

The GPR119 ligand may inhibit triglyceride accumulation in the liver.

The GPR119 ligand may increase activity of AMP-activated protein kinase (AMPK).

The GPR119 ligand may inhibit activity of sterol regulatory element binding protein-1c (SREBP-1c).

The GPR119 ligand may inhibit expression of fatty acid synthase (FAS).

The GPR119 ligand may inhibit expression of acetyl CoA carboxylase (ACC).

The GPR119 ligand may inhibit expression of stearoyl-CoA desaturase (SCD).

The non-alcoholic fatty liver disease may be selected from the group consisting of simple fatty liver, non-alcoholic steatohepatitis, liver fibrosis, and cirrhosis.

Another aspect of the present invention provides a method of preventing or treating non-alcoholic fatty liver disease, the method including a step of administering a G protein coupled receptor 119 (GPR119) ligand to a subject.

Still another aspect of the present invention provides use of a GPR119 ligand for preventing or treating non-alcoholic fatty liver disease.

### Advantageous Effects

A GPR119 ligand, as an antidiabetic, is currently being tested in clinical trials and is considered as a future key drug. Global pharmaceutical companies have made a huge investment therein. However, it was reported that a GPR119 receptor hardly appeared in the liver and fatty acid content in GPR119-deficient mice was hardly changed. Accordingly, research into a correlation between the GPR119 receptor and the non-alcoholic fatty liver was hardly conducted.

The present inventors confirmed that, when a mouse liver tissue and hepatocyte line were treated with two drugs (MBX2982 and GSK1292263) which are selective ligands for GPR119 and are current phase II clinical trial drugs, the expression of GPR119 increased, and the expressions of fatty acid synthase (FAS), acetyl CoA carboxylase (ACC), and stearoyl-CoA desaturase (SCD), which are synthesis enzymes of fatty acids and triglycerides and present in the liver, were inhibited. In addition, it was confirmed that the activity of SREBP-1c, as a key factor controlling the expression of an enzyme system synthesizing fatty acid, was inhibited by the two ligands. Further, it was confirmed that, when eight-week-old animal models fed with a high-fat diet were administered with the two ligands, fatty liver development therein was completely inhibited.

As described above, the GPR119 ligand has superior fatty liver inhibition effects, and thus, can be effectively used in preventing or treating non-alcoholic fatty liver.

### DESCRIPTION OF DRAWINGS

FIG. 1 illustrates that the expression of a GPR119 receptor is increased in human liver cancer cells treated with a GPR119 ligand.

FIG. 2 illustrates an enzyme system involved in fatty liver development [triglyceride (TG) synthesis].

FIG. 3 illustrates SREBP-1c expression inhibition effects of a GPR119 ligand.

FIG. 4 illustrates LXR reporter activity inhibition effects of a GPR119 ligand.

FIG. 5 illustrates SCD-1 and FAS mRNA expression inhibition effects of a GPR119 ligand.

FIG. 6 illustrates SREBP-1C and FAS expression inhibition effects of a GPR119 ligand upon high-glucose/high-insulin exposure.

FIG. 7 illustrates effects of a GPR119 ligand on body weight and fat accumulation in the tissues upon high-fat diet.

FIG. 8 illustrates effects of a GPR119 ligand on fatty liver development, liver weight, total serum cholesterol, glucose level, and ALT level due to high-fat diet.

FIG. 9 illustrates effects of a GPR119 ligand on mRNA expressions of SREBP-1C, FAS and GPR119 in the liver due to high-fat diet.

FIG. 10 illustrates a correlation between AMPK activity and SREBP-1C activity by a GPR119 ligand in HepG2 cells.

FIG. 11 illustrates that the function of a GPR119 ligand that affects SREBP-1C is not affected by treatment with a PKA inhibitor H-89.

FIG. 12 schematically illustrates signal pathway differences between anti-diabetic efficacy and anti-fatty liver efficacy of a GPR119 ligand.

FIG. 13 illustrates effects of a GPR119 ligand in a choline-deficient, amino acid-fixed, and high fat-dieted fatty liver mouse model.

### MODES OF THE INVENTION

The present inventors confirmed that a GPR119 ligand, which has been developed as an anti-diabetic drug, exhibits superior effects in non-alcoholic fatty liver treatment, the signal pathways in hepatocytes therefor differ from those of the small intestine and the pancreas exhibiting anti-diabetic effects, and, accordingly, the GPR119 ligand is suitable for treating non-alcoholic fatty liver, thus completing the present invention.

Hereinafter, the present invention will be described in detail.

The present invention provides a pharmaceutical composition containing a G protein coupled receptor 119 (GPR119) ligand as an effective ingredient for preventing or treating non-alcoholic fatty liver disease.

The GPR119 ligand is preferably 4-((4-(1H-tetrazol-1-yl)phenoxy)methyl)-2-(1-(5-ethylpyrimidin-2-yl)piperidin-4-yl)thiazole (MBX2982) or 3-isopropyl-5-(4-(((6-(4-(methylsulfonyl)phenyl)pyridin-3-yl)oxy)methyl)piperidin-1-yl)-1,2,4-oxadiazole (GSK1292263), but the present invention is not limited thereto. The GPR119 ligand may be one commercially available or synthesized. Alternatively, the GPR119 ligand may be any substance that binds to a GPR119 receptor and increases the expression thereof.

The GPR119 ligand is preferably a ligand which inhibits triglyceride accumulation in the liver by increasing the activity of AMP-activated protein kinase (AMPK), inhibiting the activity of sterol regulatory element binding protein-1c (SREBP-1c), and inhibiting the expression of fatty acid synthase (FAS), acetyl CoA carboxylase (ACC), or stearoyl-CoA desaturase (SCD), but the present invention is not limited thereto.

The non-alcoholic fatty liver disease includes primary and secondary non-alcoholic fatty liver diseases. Preferably, the non-alcoholic fatty liver disease refers to a non-alcoholic fatty liver disease caused by primary hyperlipidemia, diabetes, or obesity. For example, the non-alcoholic fatty liver disease includes simple fatty liver, non-alcoholic steatohepatitis, liver fibrosis, and cirrhosis.

A composition of the present invention may further include at least one publicly known effective ingredient having therapeutic effects on non-alcoholic fatty liver, along with the GPR119 ligand.

The composition of the present invention may further include a suitable carrier, vehicle and diluent commonly used to prepare a pharmaceutical composition. The composition may be formulated into oral formulations, such as a powder, a granule, a tablet, a capsule, a suspension, an emulsion, a syrup, and an aerosol, an external preparation, a suppository and a sterilized injection solution according to general methods. Suitable formulations known in the art are disclosed in the following document: Remington's Pharmaceutical Science, recent version, Mack Publishing Company, Easton Pa. Formulations disclosed in this document are preferred.

Examples of a carrier, a vehicle, and a diluent which may be included in the composition include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oils, and the like. The composition is formulated using a generally used diluent or vehicle such as a filler, an extender, a binder, a wetting agent, a disintegrant, or a surfactant.

Examples of a solid formulation for oral administration include a tablet, a pill, a powder, a granule, a capsule, etc. The composition may be prepared in such a solid formulation by mixing the same with at least one vehicle, e.g., starch, calcium carbonate, sucrose, lactose, gelatin, etc. Other than simple vehicles, a lubricant such as magnesium stearate or talc may be used. Examples of a liquid formulation for oral administration include a suspension, an elixir, an emulsion, a syrup, etc. In addition, examples of the liquid formulation may include, other than water and liquid paraffin which are commonly used simple diluents, various vehicles, e.g. a wetting agent, a sweetener, a flavoring agent, a preservative, etc. Examples of a formulation for parenteral administration include a sterilized aqueous solution, a nonaqueous solvent, a suspension, an emulsion, a lyophilized formulation, and a suppository. In addition, a suspension, as a nonaqueous solvent, may be a vegetable oil such as propylene glycol, polyethylene glycol, or olive oil, injectable ester such as ethyl oleate, etc. A base of the suppository may be Witepsol, Macrogol, Tween 61, cacao butter, laurinum, glycerogelatin, etc.

The term “administration” used in the present invention refers to provision of the composition of the present invention to a subject according to a predetermined suitable method.

A preferred administration amount of the pharmaceutical composition of the present invention depends upon the physical conditions and body weights of subjects, disease development degrees, formulation types, administration pathways, and administration periods, and may be suitably determined by those skilled in the art. To exhibit preferred effects, the GPR119 ligand of the present invention may be administered in an amount of 0.1 mg/kg to 100 mg/kg a day, preferably 1 to 30 mg/kg a day, and may be administrated once or as several doses per day.

The pharmaceutical composition of the present invention may be administrated through various paths. All paths for administration may be anticipated. For example, the composition may be administrated by oral, rectal, intravenous, intramuscular, subcutaneous, intrauterine dura mater, or cerebrovascular injection. Application of the pharmaceutical composition of the present invention depends upon drug types, as active ingredients, along with various factors such as disease types to be treated, administration path, age, sex, and body weight of patients, and severity of disease.

Further, the pharmaceutical composition of the present invention may be used alone or in combination with surgery, hormone treatment, drug treatment, and the use of a biological-response regulator to prevent and treat a non-alcoholic fatty liver disease.

Now, the present invention will be described in more detail with reference to the following preferred examples. These examples are provided for illustrative purposes only and should not be construed as limiting the scope and spirit of the present invention.

### Materials and Methods

A. Animal Experiments

Raising of Experimental Animals

In the present invention, a high fat diet (HFD: 60% fat calories, Research diets, D12492, US) was used as an obesity induction diet.

Six-week-old male C57BL/6J mice (Central Lab. Animal Inc., Seoul, Korea) were acclimated to an experimental environment for one week while being fed solid food, and then randomly divided into a control group and an experimental group for a high-fat diet using a randomized block design. The animal groups were fed for 12 weeks, and thus, fatty liver animal models were established. Fatty liver development was determined according to blood biochemical and histochemical analysis methods. The mice were orally fed with the high-fat diet only for six weeks, and in combination with a GPR119 ligand (10 mg/kg) suspended in 40% PEG400 once a day for five days every week. Choline-deficient, amino acid-fixed high-fat diet steatohepatitis models were prepared by feeding six-week-old male C57BL/6J mice (Central Lab. Animal Inc., Seoul) with a diet containing 60 kcal fat and 0.1% methionine for four weeks. After terminating the experiment, experimental animals fasted for 12 hours or more. Subsequently, blood, liver, and adipose tissues in intestines (epididymal fat, kidney-surrounding fat) were collected from the animals that had been anesthetized with diethyl ether and then washed with a 0.1 M phosphate buffered saline (pH 7.4), followed by being weighed. Blood collected from abdominal aortas was centrifuged at 3000 rpm for 20 minutes using SST tubes to separate serum.

Biochemical Analysis Methods for Blood and Liver Tissues

A total cholesterol concentration and a total glucose concentration in serum from each of the aforementioned experimental animals raised for 12 weeks were measured as follows. Each of the total cholesterol concentration and the total glucose concentration in serum was measured twice by means of a commercial measurement kit (Bio Clinical Systems). The amount of alanine aminotransferase (ALT) in blood used as a liver function index was measured by means of a commercial analysis kit (Bio Clinical Systems, Korea).

Hematoxylin and Eosin (H&E) Staining

The collected liver tissues were fixed with a 10% neutral formalin solution and then subjected to a common fixation procedure and dehydration process, followed by embedding the same in paraffin. The embedded tissues were sectioned to a thickness of 4 μm and stained with H&E. The stained tissues were observed by means of an optical microscope.

Investigation of Protein Expressions in Tissues by Western Blotting

A predetermined amount of each of the liver tissues was homogenized in liquid nitrogen and a cell lysis buffer in a mortar, and then a lysed tissue solution was transferred to a new tube, followed by vortexing. Centrifugation was conducted at 14,000 rpm and 4° C. for 20 minutes. A middle layer of the centrifuged solution was collected and then subjected to protein quantification by the Bradford assay. 30 μg of a protein thereof was electrophoresed on an SDS polyacrylamide gel. Expression changes of FAS and SREBP-1c proteins were measured by western blotting.

Isolation of RNA from Tissue using a TRIzol Method and Confirmation Thereof

1 ml of a TRIzol solution was added to 0.1 g of each of the liver tissues to lyse the tissue, and then centrifuged at 4° C. and 12,000×g for 10 minutes. A supernatant was transferred to a new tube and 200 μl of chloroform was added thereto, followed by vortexing. A supernatant was transferred to a new tube, and then isopropanol was added thereto in a ratio of 1:1 with the supernatant. A resultant mixture was strongly vortexed for 15 seconds and then allowed to sit for 10 minutes at room temperature. Subsequently, centrifugation was performed at 12,000×g and 4° C. for 10 minutes to remove a supernatant from the mixture, and then 1 ml of 70% ethanol was added to the remaining precipitate, followed by centrifuging at 7,500×g and 4° C. for five minutes. After removal of the ethanol, the tube containing an RNA precipitate was dried for five minutes at room temperature and an RNA pellet was dissolved in nuclease free water. The concentrations of extracted RNA samples were measured by means of a UV/VIS spectrum analyzer (Nanodrop, Thermo, US) at wavelengths of 260 nm and 280 nm. cDNAs were synthesized using an RT kit, and then mRNA expression changes of SCD-1 and FAS were investigated by real time PCR.

mRNA Expression Analysis by Real Time-Polymerase Chain Reaction (Real Time-PCR)

The RNA sample extracted from each of the liver tissues was reversely transcribed using a cDNA synthesis PCR kit to synthesize cDNAs. The cDNAs obtained through reverse transcription were used as a template and primers for 5′ and 3′ flanking sequences of the cDNAs were used as shown in [Table 1] below. Real time PCR (Mini-Opticon, Bio-Rad, US) was carried out and the expression levels of mRNA were investigated.

TABLE 1

| | | | Anneal- | | |
|---|---|---|---|---|---|
| | | | ing | | PCR |
| | | | temper- | Se- | pro- |
| | Pri- | | ature | quence | duct |
| Gene | mer | Sequence (5′-3′) | (□) | No. | (bp) |
| GPR119 | F | TGCAGCTGCCTCTGTCCTCA | 64.2 | 1 | 252 |
| | R | GCACAGGAGAGGGTCAGCAC | 61.8 | 2 | bp |
| β- | F | CCACAGCTGAGAGGGAAATC | 58.2 | 3 | 193 |
| actin | R | AAGGAAGGCTGGAAAAGAGC | 58.5 | 4 | bp |

B. Isolation of Hepatocytes from Mice and Culturing the Same

Liver cells were collected from male C57BL/6 mice (Central Lab. Animal Inc., Seoul) according to a method by Seglen et al. (Exp Cell Res., 82, pp 391-398, 1973). Each C57BL/6 mouse was anesthetized and then the abdomen thereof was incised. Subsequently, the hepatic portal vein was intubated and a gas mixture of 5% CO₂ and 95% O₂ was insufflated thereinto. A Ca²⁺ and Mg²⁺-free Hank's balanced salt solution at 37° C. was flowed into the tube to remove blood in the liver. Subsequently, a 0.1% collagenase type IV (Sigma, US) solution was flowed thereinto. The liver tissue was removed from the body, and then prepared into a hepatocyte suspension. The prepared hepatocyte suspension was centrifuged at 50×g for two minutes. Precipitated hepatocytes were collected and washed with a Ca²⁺ and Mg²⁺-free Hank's balanced salt solution twice. The washed cells were cultured in a number of 5×10⁶ cells/ml in a culture plate coated with collagen type I (Sigma, US). A culture medium thereof was Williams Medium E (WME, GibcoBRL, USA) including 10% (v/v) fetal bovine serum (FBS, GibcoBRL), 10-8M insulin, 50 U/ml of penicillin (Sigma), and 50 μg/ml of streptomycin (Sigma). Culture conditions thereof were 5% CO₂ and 37° C. After culturing for one hour, cells were washed with Hank's balanced salt solution (HBSS) containing Ca²⁺ and Mg²⁺ to remove detached hepatocytes, and the cells were incubated in a new medium under conditions of 5% CO₂ and 37° C. for five hours. Subsequently, the medium was replaced by a medium not containing FBS. After additionally culturing for 18 hours, the cells were used as a sample for an experiment.

C. Culturing of Human Hepatocyte Line (HepG2)

A 6-well plate was inoculated with HepG2 (ATCC, US), as a human hepatocyte line, and cultured until confluence in a DMEM medium containing 1% penicillin-streptomycin (Hyclone, US) and 10% fetal bovine serum (Hyclone, US) in a 5% CO₂ incubator at 37° C. The medium containing 10% fetal bovine serum of the hepatocyte line grown to confluence was replaced by a medium not containing FBS. The cell line was additionally cultured for 18 hours and used as a sample for an experiment.

D. Western Blot

Sodium dodecyl sulfate-polyacrylamide gel electrophoresis (SDS-PAGE) was carried out by means of a gel electrophoresis device (Mighty Small SE 250, Hoefer Scientific Instruments, San Francisco) according to a method by Laemmli (UK, Nature 227, 680-685, 1970). Each of cell lysate fractions was diluted with a buffer solution for sample dilution [63 mM Tris (pH.6.8), 10% glycerol, 2% SDS, 0.0013% bromophenol blue, and 5% β-mercaptoethanol], and then electrophoresed on a 8% or 10% gel in an electrode buffer solution (1 l of the solution contained 15 g of Tris, 72 g of glycerin, and 5 g of SDS). Proteins of the electrophoresed gel were transferred to a nitrocellulose membrane over three hours in a potential buffer solution (25 mM Tris, 192 mM glycerin, and 20% v/v methanol (pH.8.3)] by means of an electrophoresis device for transferring proteins at 40 mA. Each of anti-fatty acid synthase (anti-FAS), anti-GPR119, anti-SREBP-1c, anti-phosphoric acid AMPK-α, anti-phosphoric acid ACC, and anti-phosphoric acid SREBP-1c, as a primary antibody, was reacted with the nitrocellulose membrane, and then the nitrocellulose membrane was reacted with horseradish peroxidase-conjugated goat anti-rabbit IgG and horseradish peroxidase-conjugated goat anti-mouse IgG, as secondary antibodies, for one hour. Chemiluminescence detection was performed using an ECL chemiluminescence system (Amersham, Gaithesburg, Mass.). Whether total protein contents in samples are the same is determined using anti-β-actin and anti-LaminA/C antibodies. Changes in protein expression amounts were determined by measuring the intensities of chemiluminescence signals on the blot by means of densitometry. Upon scanning with a densitometer, an image scan and an analysis system (Alpha-Innotech Co.) were used. Each lane was calculated using AlphaEase™ version 5.5 software and background intensity was removed from the calculated values.

E. Isolation of RNAs from Cells using TRIzol Method and Confirmation of the Isolated RNAs

1 ml of a TRIzol solution was added to each of cell samples to lyse the cells. Subsequently, centrifugation was carried out at 4° C. and 12,000×g for 10 minutes. A supernatant was transferred to a new tube and 200 μl of chloroform was added thereto, followed by vortexing. A supernatant was transferred to a new tube and then isopropanol was added thereto in the same amount as the supernatant. After strongly vortexing for 15 seconds, the tube was allowed to sit at room temperature for 10 minutes and then centrifuged at 12,000×g and 4° C. for 10 minutes. Subsequently, a supernatant was removed from the tube and then 1 ml of 70% ethanol was added to the remaining precipitate, followed by centrifuging at 7,500×g and 4° C. for five minutes. After removing the ethanol, the tube containing an RNA precipitate was dried at room temperature for five minutes. A formed RNA pellet was dissolved in nuclease free water. The concentration of an extracted RNA sample was measured at wavelengths of 260 nm and 280 nm by means of a UV/VIS spectrum analyzer (Nanodrop, Thermo, US) and cDNAs were synthesized therefrom using an RT kit. Subsequently, mRNA expression changes of SCD-1 and FAS were investigated using real time PCR.

F. Analysis of mRNA Expressions in Cells using Real Time-PCR

The RNA samples extracted from cells were reversely transcribed using a cDNA synthesis PCR kit to synthesize cDNAs from each thereof. The cDNAs obtained by reverse transcription were used as templates and primers for 5′ and 3′ flanking sequences of the cDNAs were used as shown in [Table 1] below. Real time PCR (Mini-Opticon, Bio-Rad, US) was carried out and the expression levels of mRNA were investigated.

TABLE 2

| | | | Anneal- | | |
|---|---|---|---|---|---|
| | | | ing | | PCR |
| | | | temper- | Se- | pro- |
| | Pri- | | ature | quence | duct |
| Gene | mer | Sequence (5′-3′) | (□) | No. | (bp) |
| SCD1 | F | GCTGCTCGGATCACTAGTGAA | 61.1 | 5 | 281 |
| | R | TTCTGCTATCAGTCTGTCCAG | 65.4 | 6 | bp |
| FAS | F | AGTACACACCCAAGGCCAAG | 65 | 7 | 125 |
| | R | GGATACTTTCCCGTCGCATA | 62 | 8 | bp |
| β- | F | GATGAGATTGGCATGGCTTT | 61 | 9 | 102 |
| actin | R | GTCACCTTCACCGTTCCAGT | 65 | 10 | bp |

### EXAMPLE

### Example 1

### Expression Increase of GPR199 Receptor by GPR199 Ligand in Human Hepatocyte Line

In conventional studies, GPR119 expression in the liver was hardly observed. Accordingly, the function of a GPR119 ligand on fatty liver was not evaluated (Odori S et al., Metabolism, in press).

Accordingly, the present inventors confirmed that, when HepG2 (human liver cancer cell line) cells were treated with each of two selective GPR119 ligand types, i.e., MBX2982 and GSK1292263, GPR119 expression thereof was changed.

To perform this, a human hepatocyte line (HepG2) cultured according to the aforementioned methods B and C was treated with a 3 μM GPR119 ligand (MBX-2982, GSK-1292263A) in a time-dependent manner. Here, treatment conditions with the GPR119 ligand of each experimental group are summarized in [Table 3] and [Table 4] below. Expression level changes of the GPR119 protein were measured using the western blotting method of the aforementioned method D.

TABLE 3
Human hepatocyte line (HepG2)

| Classification | Treatment method |
|---|---|
| Group 1 | Treatment with normal medium |
| Group 2 | Treatment with medium containing 3 μM GPR119 ligand |
| | (MBX-2982) for one hour |
| Group 3 | Treatment with medium containing 3 μM GPR119 ligand |
| | (MBX-2982) for three hours |
| Group 4 | Treatment with medium containing 3 μM GPR119 ligand |
| | (MBX-2982) for six hours |
| Group 5 | Treatment with medium containing 3 μM GPR119 ligand |
| | (MBX-2982) for nine hours |

TABLE 4
Human hepatocyte line (HepG2)

| Classification | Treatment method |
|---|---|
| Group 1 | Treatment with normal medium |
| Group 2 | Treatment with medium containing 3 μM GPR119 ligand |
| | (GSK-1292263A) for 0.5 hours |
| Group 3 | Treatment with medium containing 3 μM GPR119 ligand |
| | (GSK-1292263A) for one hour |
| Group 4 | Treatment with medium containing 3 μM GPR119 ligand |
| | (GSK-1292263A) for three hours |
| Group 5 | Treatment with medium containing 3 μM GPR119 ligand |
| | (GSK-1292263A) for six hours |
| Group 6 | Treatment with medium containing 3 μM GPR119 ligand |
| | (GSK-1292263A) for nine hours |

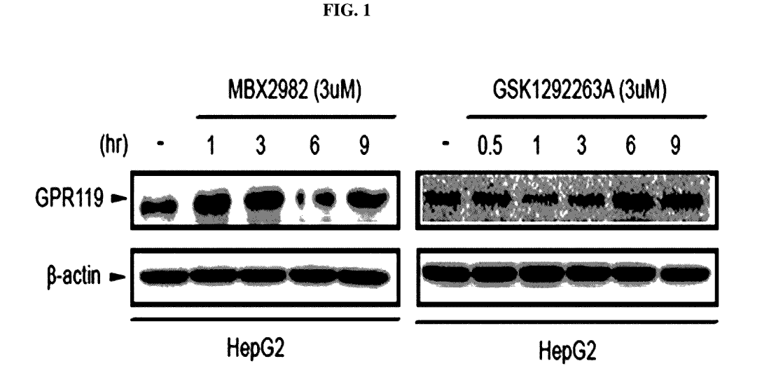

As results, the two selective ligand types of GPR119 triggered GPR119 expression increase in HepG2 cells (FIG. 1).

### Example 2

### Inhibition of SREBP-1c Activity and Fatty Acid Synthesis Enzyme Expression by GPR199 Ligand in Human Hepatocyte Line and Primary Mice Hepatocytes

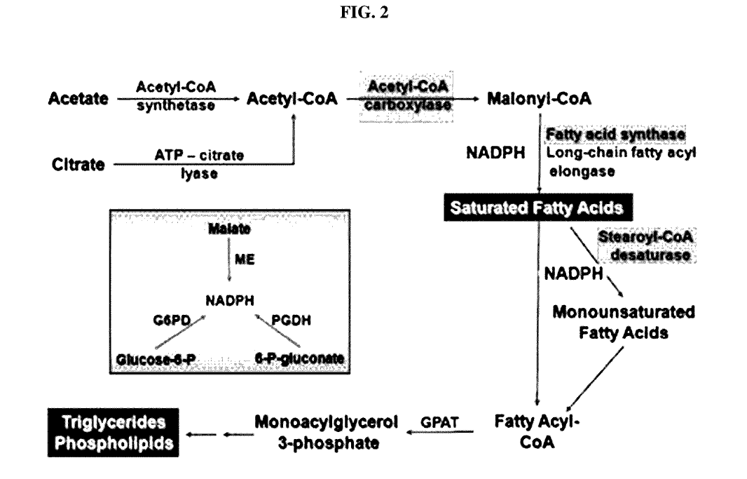

Fatty acid synthesis involved in fatty liver development is related to expression increase in a series of an enzyme system as illustrated in FIG. 2. The expressions of acetyl-CoA carboxylase (ACC), fatty acid synthase (FAS), and stearoyl-CoA desaturase (SCD) of the enzyme system are controlled by a transcriptional factor, sterol regulatory element binding protein-1c (SREBP-1c). Activation of SREBP-1c causes expression of the enzyme system, thus facilitating the accumulation of triglycerides in the liver.

2-1. Investigation of SREBP-1c Expression change by T0901317 Treatment

To investigate fat accumulation inhibition effects in the hepatocyte line treated with a GPR119 ligand, the present inventors first used a liver X receptor (LXR) ligand, i.e., T0901317 (N-(2,2,2-Trifluoroethyl)-N-[4-[2,2,2-trifluoro-1-hydroxy-1-(trifluoromethyl) ethyl] phenyl] benzenesulfonamide), known as an SREBPP-1c activation signal.

Particularly, a medium containing a GPR119 ligand (MBX-2982, GSK-1292263A) was added to hepatocytes, which were isolated and cultured as in the method B, and a human hepatocyte line (HepG2), which was cultured as in the method C. 30 minutes later, the cells were treated with 10 μM T0901317 for nine hours. Here, treatment conditions with the GPR119 ligand for each experimental group are summarized in [Table 5] and [Table 6] below. Expression change of the SREBP-1c protein was measured using the western blotting method of the method D.

TABLE 5
Primary hepatocytes

| Classification | Treatment method |
|---|---|
| Group 1 | Treatment with normal medium |
| Group 2 | Treatment with medium containing 10 μM T0901317 |
| Group 3 | Treatment with medium containing 10 μM T0901317 |
| | and 0.3 μM GPR119 ligand |
| Group 4 | Treatment with medium containing 10 μM T0901317 |
| | and 1 μM GPR119 ligand |
| Group 5 | Treatment with medium containing 10 μM T0901317 |
| | and 3 μM GPR119 ligand |
| Group 6 | Treatment with medium containing 10 μM T0901317 |
| | and 10 μM GPR119 ligand |

TABLE 6
Human hepatocyte line (HepG2)

| Classification | Treatment method |
|---|---|
| Group 1 | Treatment with normal medium |
| Group 2 | Treatment with medium containing 10 μM T0901317 |
| Group 3 | Treatment with medium containing 10 μM T0901317 |
| | and 0.1 μM GPR119 ligand |
| Group 4 | Treatment with medium containing 10 μM T0901317 |
| | and 0.3 μM GPR119 ligand |
| Group 5 | Treatment with medium containing 10 μM T0901317 |
| | and 1 μM GPR119 ligand |
| Group 6 | Treatment with medium containing 10 μM T0901317 |
| | and 3 μM GPR119 ligand |

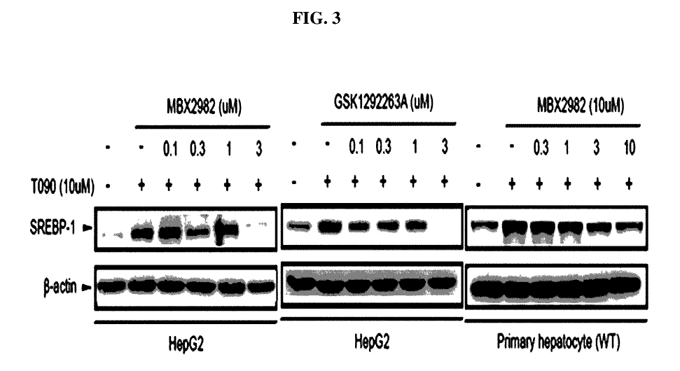

As a result, it can be confirmed that, when HepG2 and primary hepatocytes are treated with a liver X receptor (LXR) ligand alone, i.e., T0901317, known as an SREBP-1c activation signal, the expression level of SREBP-1c greatly increases compared to the case in which treatment is not performed, but, when any one of the two GPR119 ligand types is treated with T0901317, SREBP-1c expression is inhibited in a concentration-dependent manner (FIG. 3).

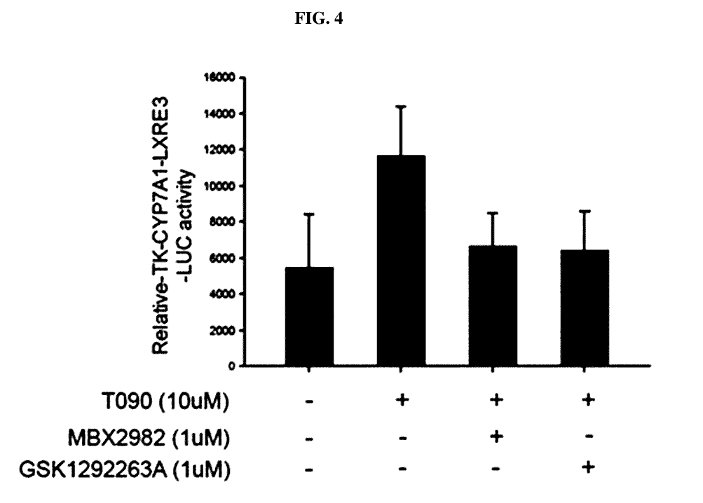

In addition, it can be confirmed that, also in LXR activity evaluation by a specific reporter gene assay, LXR activity is inhibited when T0901317 is treated with any one of the two GPR119 ligands (FIG. 4).

2-2. Investigation of mRNA Expression Level Change in SCD-1 and FAS by T0901317 Treatment

A medium containing the GPR119 ligand (MBX-2982, GSK-1292263A) was added to a human hepatocyte line (HepG2) cultured as in the method C and, 30 minutes later, the cell line was treated with 10 μM T0901317 for nine hours. Treatment conditions with the GPR119 ligand for each experimental group are summarized in [Table 7] below. To investigate mRNA expression change in SCD-1 and FAS, RNAs were isolated and cDNAs were synthesized therefrom as in the method E. mRNA expression levels were measured using real time-PCR as in the method F.

TABLE 7
Human hepatocyte line (HepG2)

| Classification | Treatment method |
|---|---|
| Group 1 | Treatment with normal medium |
| Group 2 | Treatment with medium containing 10 μM T0901317 |
| Group 3 | Treatment with medium containing 10 μM T0901317 |
| | and 0.1 μM GPR119 ligand |
| Group 4 | Treatment with medium containing 10 μM T0901317 |
| | and 0.3 μM GPR119 ligand |
| Group 5 | Treatment with medium containing 10 μM T0901317 |
| | and 1 μM GPR119 ligand |
| Group 6 | Treatment with medium containing 10 uM T0901317 |
| | and 3 uM GPR119 ligand |

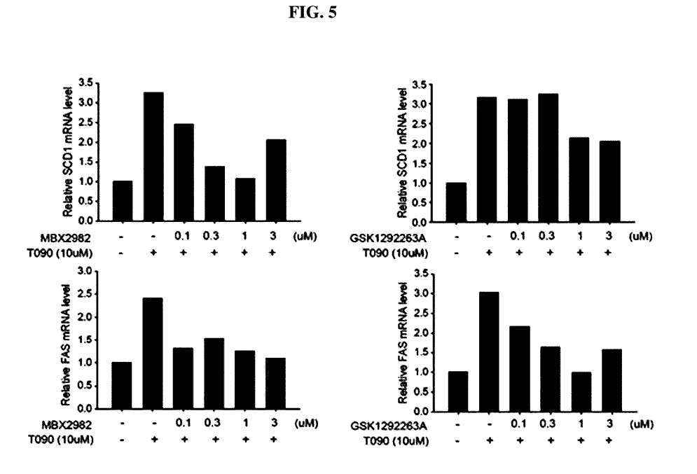

As a result, it can be confirmed that, with regard to mRNA expressions of SCD-1 and FAS involved in triglyceride synthesis in the liver, increased mRNA expression levels of SCD-1 and FAS due to treatment with T0901317 are decreased in a concentration-dependent manner upon treatment with the two GPR119 ligand types (FIG. 5).

2-3. Investigation of SREBP-1c and FAS Expression Change According to High-Glucose/High-Insulin Exposure

To investigate SREBP-1c and FAS expression change according to, other than exposure to the LXR ligand, exposure to high-glucose/high-insulin known as signals causing non-alcoholic fatty liver, HepG2 and primary human hepatocytes were exposed to high-glucose/high-insulin and, in this state, the cells were treated with the two GPR119 ligand types.

Particularly, a medium containing high glucose (30 mM glucose) was added to the human hepatocyte line (HepG2) and the cell line was treated with the GPR119 ligand in a concentration-dependent manner. After 30 minutes, the cell line was treated with 200 nM insulin for 24 hours. Here, treatment conditions with the GPR119 ligand for each experimental group are summarized in [Table 8] and [Table 9] below. Protein expression change therein was measured using the western blotting method as in the method D.

TABLE 8
Primary hepatocytes

| Classification | Treatment method |
|---|---|
| Group 1 | Treatment with normal medium |
| Group 2 | Treatment with high-glucose and high-insulin medium |
| Group 3 | Treatment with high-glucose and high-insulin medium |
| | and treatment with medium containing 0.3 μM |
| | GPR119 ligand |
| Group 4 | Treatment with high-glucose and high-insulin medium |
| | and treatment with medium containing 1 μM |
| | GPR119 ligand |
| Group 4 | Treatment with high-glucose and high-insulin medium |
| | and treatment with medium containing 3 μM GPR119 |
| | ligand |
| Group 4 | Treatment with high-glucose and high-insulin medium |
| | and Treatment with medium containing 10 μM |
| | GPR119 ligand |

TABLE 9
Human hepatocyte line (HepG2)

| Classification | Treatment method |
|---|---|
| Group 1 | Treatment with normal medium |
| Group 2 | Treatment with high-glucose and high-insulin medium |
| Group 3 | Treatment with high-glucose and high-insulin medium |
| | and treatment with medium containing 1 μM GPR119 |
| | ligand |
| Group 4 | Treatment with high-glucose and high-insulin medium |
| | and treatment with medium containing 3 μM GPR119 |
| | ligand |

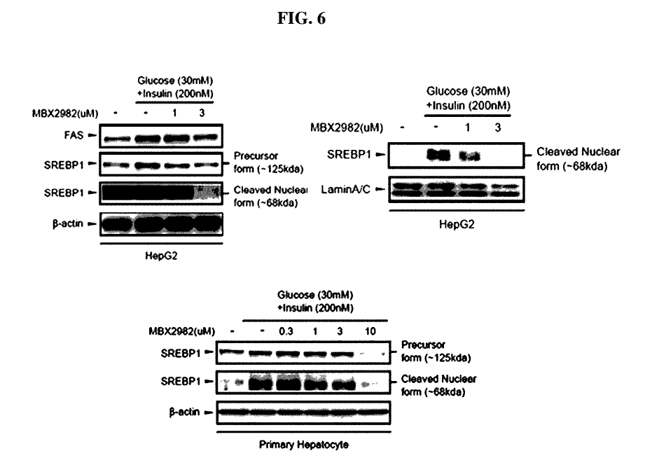

As a result, it can be confirmed that, when HepG2 and the primary human hepatocytes are exposed to high-glucose/high-insulin, the expression levels of SREBP-1c and nuclear migration-type (active-type) SREBP-1C in each thereof remarkably increase. In addition, it can be confirmed that such increased SREBP-1c expressions are completely inhibited upon treatment with MBX2982 as a GPR119 ligand (FIG. 6). Further, it can be confirmed that, in the HepG2 cells, increased FAS expression due to high-glucose/high-insulin exposure is also inhibited by treatment with MBX2982 (FIG. 6).

### Example 3

### Efficacy of GPR199 Ligand in Fatty Liver Model Induced by High-Fat Diet

To investigate whether the GPR119 ligands inhibited fatty liver development, the effects of the GPR119 ligand were investigated in a fatty liver model induced by a high-fat diet based on the animal experiment method of the method A.

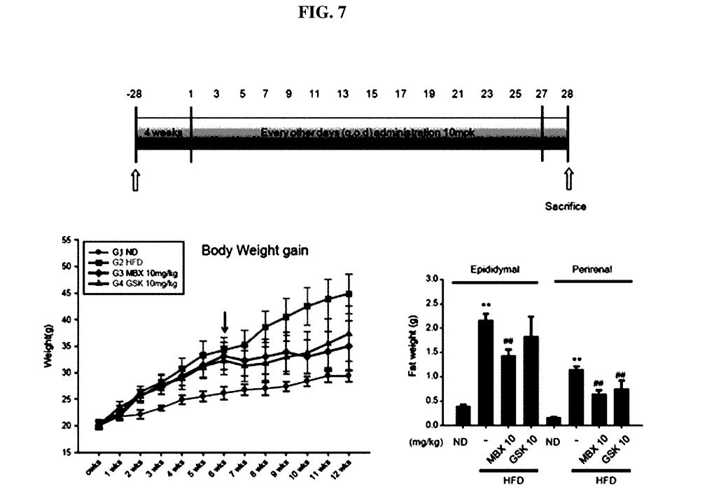

Particularly, animals were fed with a high-fat diet for four weeks and then orally administered with the two drug types, the amount of each of which was 10 mg/kg, once per two days for additional four weeks under the high-fat diet condition. A particular experimental schedule is illustrated at an upper part of FIG. 7.

3-1. Investigation of Body Weight Decrease Effect

It was investigated whether, in the fatty live model induced by a high-fat diet, the body weights of the animals were reduced due to treatment with the GPR119 ligand.

As a result, it can be confirmed that the body weights of the animals fed with a high-fat diet are remarkably increased and the body weights of the animals treated with the GPR119 ligand are significantly decreased (FIG. 7). In addition, it can be confirmed that, in the case of the group treated with MBX2982, the fat weight in the tissue is significantly decreased.

3-2. Investigation of Fat Accumulation Degree by H&E Staining

Liver tissues were extracted and then subjected to H&E staining to observe a fat accumulation degree.

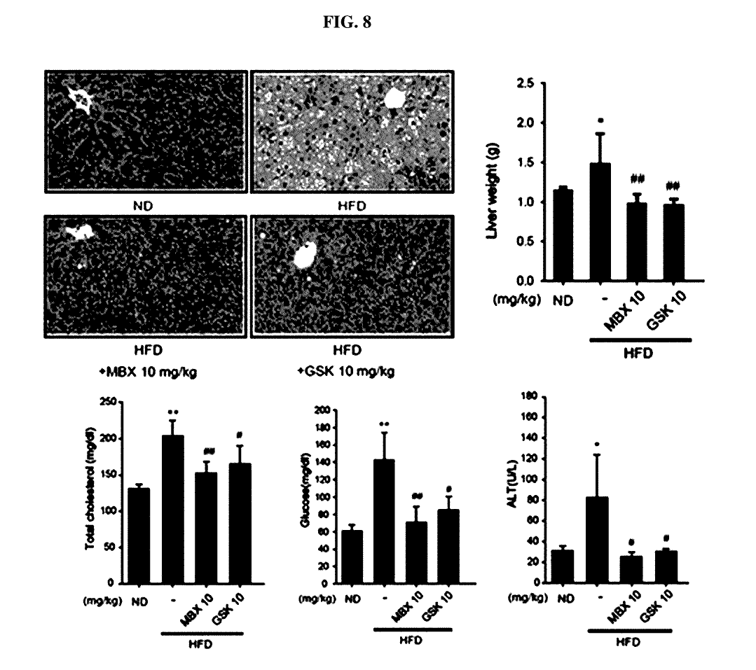

As a result, while fat accumulation was observed in hepatocytes of most animals of the group treated with a high-fat diet, the fat accumulation was remarkably improved in the group treated with the GPR119 ligand (FIG. 8). In addition, a liver weight, a total cholesterol amount, a blood glucose level, and an ALT level were increased in the group with a high-fat diet, but remarkably reduced in the group treated with the GPR119 ligand. Accordingly, the anti-fat-accumulation efficacy of the GPR119 ligand was confirmed (FIG. 8).

3-3. Investigation of SREBP-1C and FAS Expression in Liver Tissue

SREBP-1C and FAS expressions in the liver tissue of the fatty liver model induced by a high-fat diet were investigated.

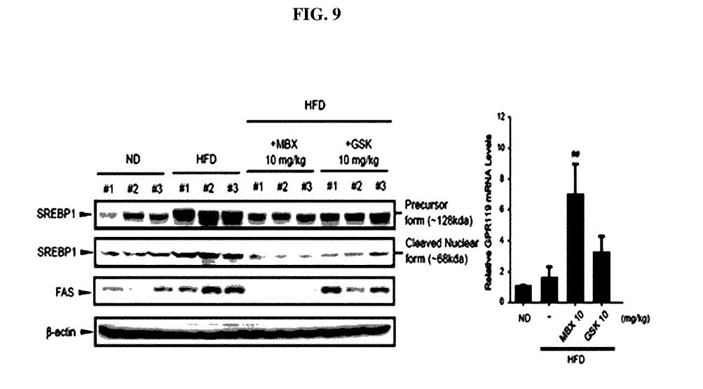

As a result, it can be confirmed that, in the group treated with the GPR119 ligand, both SREBP-1C expression and FAS expression tend to be inhibited, compared to the group treated with a high-fat diet (FIG. 9).

In addition, when the two GRP119 ligand types were administered to mice, the mRNA levels of the ligands increased. In particular, upon administration with MBX2982, the mRNA level of GPR119 seven-fold increased, compared to a control group.

### Example 4

### Function of GPR119 Ligand as AMPK Activator

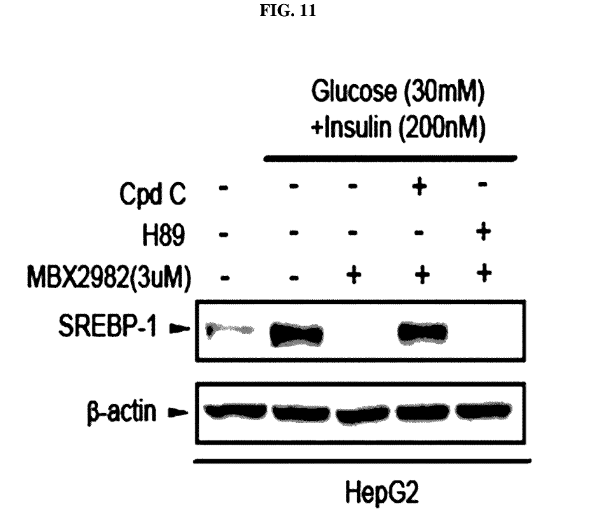

The present inventors inferred that, based on the aforementioned results, the activation of SREBP-1C by the GPR119 ligand is not related to cAMP/PKA (cyclic AMP/Protein kinase A) activation known as a secondary signal system of GPR119. Accordingly, the present inventors confirmed that the function of the GPR119 ligand affecting SREBP-1C was not affected by treatment with H-89 as a PKA inhibitor (FIG. 11).

An experiment therefor was performed as follows. A medium containing high glucose (30 mM) was added to the human hepatocyte line (HepG2) and then the cell line was treated with an AMPK inhibitor. After 30 minutes, the cell line was treated with the GPR119 ligand at a concentration of 3 μM. After 30 minutes, the cell line was treated with 200 nM insulin and cultured for 24 hours. Treatment conditions with the GPR119 ligand for each experimental group are summarized in [Table 10] below. Protein expression change was measured using the western blotting method of the method D.

TABLE 10
Human hepatocyte line (HepG2)

| Classification | Treatment method |
|---|---|
| Group 1 | Treatment with normal medium |
| Group 2 | Treatment with high-glucose and high-insulin medium |
| Group 3 | Treatment with high-glucose and high-insulin medium |
| | and treatment with medium containing 3 μM GPR119 |
| | ligand |
| Group 4 | Treatment with high-glucose and high-insulin medium |
| | and treatment with medium containing AMPK inhibitor |
| | and 3 μM GPR119 ligand |
| Group 5 | Treatment with high-glucose and high-insulin medium |
| | and treatment with medium containing PKA inhibitor |
| | and 3 μM GPR119 ligand |

Recently, it was reported that a ser-372 region of SREBP-1C was phosphorylated by AMP-activated protein kinase (AMPK), whereby SREBP-1C was inactivated. Accordingly, the present inventors evaluated the influence of GPR119 ligands on AMPK activation and SREBP-1C phosphorylation in hepatocytes.

To perform this evaluation, a human hepatocyte line (HepG2) cultured as in the methods B and C was treated with the GPR119 ligand (MBX-2982, GSK-1292263A) at a concentration of 3 μM in a time-dependent manner. Here, treatment conditions with the GPR119 ligand for each experimental group are summarized in [Table 11] and [Table 12] below. Protein expression change was measured using the western blotting method of the method D.

TABLE 11
Human hepatocyte line (HepG2)

| Classification | Treatment method |
|---|---|
| Group 1 | Treatment with normal medium |
| Group 2 | Treatment with medium containing 3 μM GPR119 ligand |
| | (MBX-2982) for one hour |
| Group 3 | Treatment with medium containing 3 μM GPR119 ligand |
| | (MBX-2982) for three hours |
| Group 4 | Treatment with medium containing 3 μM GPR119 ligand |
| | (MBX-2982) for six hours |
| Group 5 | Treatment with medium containing 3 μM GPR119 ligand |
| | (MBX-2982) for nine hours |

TABLE 12
Human hepatocyte line (HepG2)

| Classification | Treatment method |
|---|---|
| Group 1 | Treatment with normal medium |
| Group 2 | Treatment with medium containing 3 μM GPR119 ligand |
| | (GSK-1292263A) for 0.5 hours |
| Group 3 | Treatment with medium containing 3 μM GPR119 ligand |
| | (GSK-1292263A) for one hour |
| Group 4 | Treatment with medium containing 3 μM GPR119 ligand |
| | (GSK-1292263A) for three hours |
| Group 5 | Treatment with medium containing 3 μM GPR119 ligand |
| | (GSK-1292263A) for six hours |
| Group 6 | Treatment with medium containing 3 μM GPR119 ligand |
| | (GSK-1292263A) for nine hours |

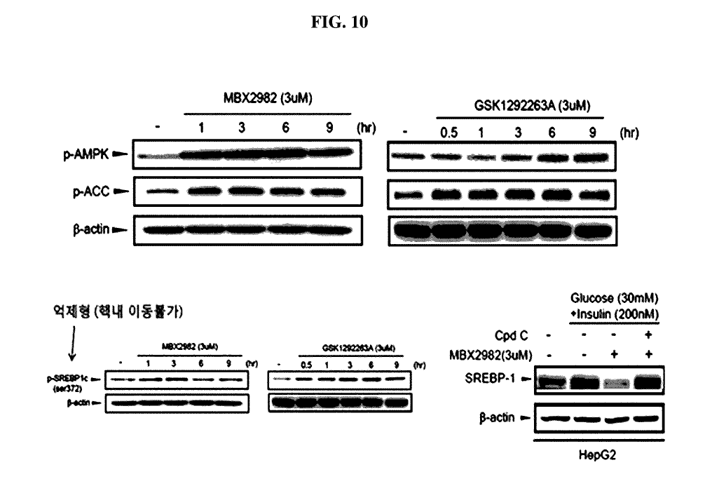

As a result, it can be confirmed that, in the hepatocytes, all of the two GPR119 ligand types strongly activate AMPK (AMPK phosphorylation and ACC phosphorylation) and, in this case, phosphorylation of ser-372 of SREBP-1C increases (FIG. 10). Further, it can be confirmed that, upon treatment with Compound C as an AMPK inhibitor, SREBP-1C expression inhibition due to MBX2982, as a GPR119 ligand, is recovered (FIG. 10).

### Example 5

### Efficacy of GPR199 Ligand in Choline-Deficient, Amino Acid-Fixed, and High-Fat Diet-Fed Steatohepatitis Model

To investigate whether the GPR119 ligands had effects on non-alcoholic steatohepatitis, the effects of the GPR119 ligands were investigated in a choline-deficient, amino acid-fixed, and high-fat diet-fed steatohepatitis model based on the animal experiment method of the method A.

In particular, the animals were fed with a high-fat diet for four weeks and orally administered with MBX2982 in an amount of 10 mg/kg once per two days for additional four weeks under a high-fat diet condition. A particular experimental schedule therefor is illustrated at an upper part of FIG. 7.

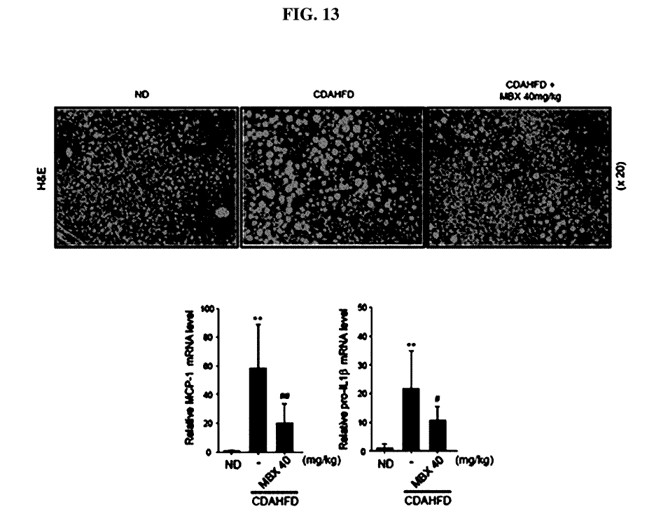

Liver tissues were extracted and subjected to H&E staining to observe a fat accumulation degree. As a result, when the amino acid-fixed and high-fat diet-fed non-alcoholic steatohepatitis model was administered with the GPR 119 ligand, fat accumulation was inhibited (FIG. 13). In addition, mRNA expressions of MCP-1 and Pro-IL-1beta, as representative inflammatory markers of steatohepatitis, were investigated. As a result, it can be confirmed that mRNA expressions of MCP-1 and Pro-IL-1beta are inhibited due to administration of the GPR 119 ligand (FIG. 13).

Accordingly, it can be confirmed that, aside from a previously known GLP-1 and insulin secretion function through cAMP increase by the GPR119 ligand, the ligands activate AMPK in hepatocytes and thus exhibit inhibition effects on fatty liver development. In addition, it can be confirmed that the GPR 119 ligand inhibits progressive steatohepatitis as well as non-alcoholic fatty liver.

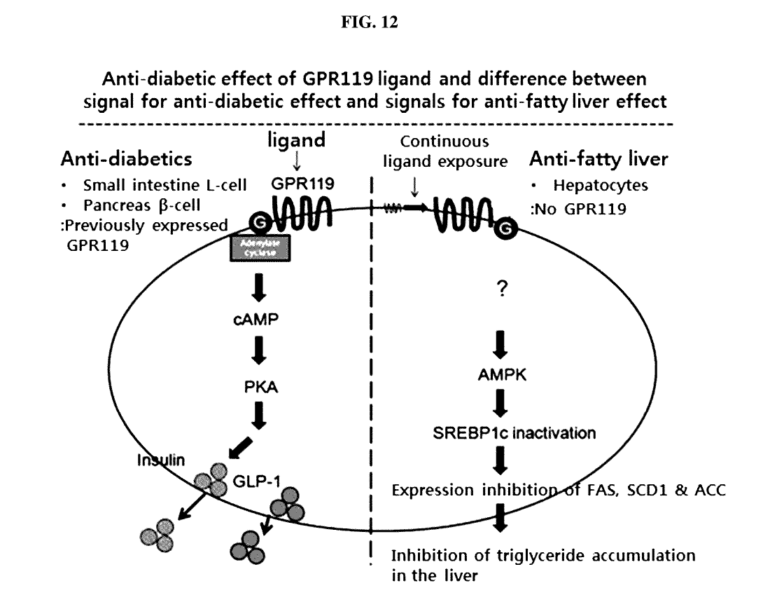

Therefore, the present invention is characterized in that the expression of the GPR119 receptor in the liver is increased due to exposure to the ligand thereof, the expressions of fatty acids and the triglyceride synthesis enzymes are inhibited upon treatment with the ligand, and, accordingly, the ligand has a therapeutic effect on fatty liver. A pharmacological mechanism thereof is related to AMPK activation, unlike previously known PKA signal activation according to cAMP increase. The pharmacological mechanism is schematically illustrated in FIG. 12. As illustrated in FIG. 12, the signal pathway for the fatty liver inhibition of the GPR119 ligand clearly differs from the signal pathway for anti-diabetic effects thereof. The present invention first confirmed the fatty liver inhibition effect of the GPR119 ligand.

The aforementioned description of the present invention is provided by way of example and those skilled in the art will understood that the present invention can be easily changed or modified into other specified forms without change or modification of the technical spirit or essential characteristics of the present invention. Therefore, it should be understood that the aforementioned examples are only provided by way of example and not provided to limit the present invention.

### INDUSTRIAL APPLICABILITY

The present inventors confirmed that, when a mouse liver tissue and hepatocyte line were treated with two drugs (MBX2982 and GSK1292263) which are selective ligands for GPR119 and are current phase II clinical trial drugs, the expression of GPR119 increased, and the expressions of fatty acid synthase (FAS), acetyl CoA carboxylase (ACC), and stearoyl-CoA desaturase (SCD), which were synthesis enzymes of fatty acids and triglycerides and present in the liver, were inhibited. In addition, it was confirmed that the activity of SREBP-1c, as a key factor controlling the expression of an enzyme system synthesizing fatty acid, was inhibited by the two ligands. Further, it was confirmed that, when eight-week-old animal models fed with a high-fat diet were administered with the two ligands, fatty liver development therein was completely inhibited.

As described above, the GPR119 ligand has superior fatty liver inhibition effects, and thus, can be effectively used in preventing or treating non-alcoholic fatty liver.

1. A method of preventing or treating non-alcoholic fatty liver disease, comprising:
administering an effective amount of a pharmaceutical composition containing a G protein coupled receptor 119 (GPR119) ligand as an active ingredient to a subject in need thereof for treating non-alcoholic fatty liver disease.

2. The method of claim 1, wherein the GPR119 ligand is 4-((4-(1H-tetrazol-1-yl)phenoxy)methyl)-2-(1-(5-ethylpyrimidin-2-yl)piperidin-4-yl)thiazole (MBX2982) or 3-isopropyl-5-(4-(((6-(4-(methylsulfonyl)phenyl)pyridin-3-yl)oxy)methyl)piperidin-1-yl)-1,2,4-oxadiazole (GSK1292263).

3. The method of claim 1, wherein the GPR119 ligand inhibits triglyceride accumulation in the liver.

4. The method of claim 1, wherein the GPR119 ligand increases activity of AMP-activated protein kinase (AMPK).

5. The method of claim 1, wherein the GPR119 ligand inhibits activity of sterol regulatory element binding protein-1c (SREBP-1c).

6. The method of claim 1, wherein the GPR119 ligand inhibits expression of fatty acid synthase (FAS).

7. The method of claim 1, wherein the GPR119 ligand inhibits expression of acetyl CoA carboxylase (ACC).

8. The method of claim 1, wherein the GPR119 ligand inhibits expression of stearoyl-CoA desaturase (SCD).

9. The method of claim 1, wherein the non-alcoholic fatty liver disease is selected from the group consisting of simple fatty liver, non-alcoholic steatohepatitis, liver fibrosis, and cirrhosis.

10. A method of inhibiting triglyceride synthesis in the liver, comprising:
administering an effective amount of a pharmaceutical composition containing a G protein coupled receptor 119 (GPR119) ligand as an active ingredient to a subject in need thereof for inhibiting triglyceride synthesis in the liver.

11. The method of claim 10, wherein the GPR119 ligand is 4-((4-(1H-tetrazol-1-yl)phenoxy)methyl)-2-(1-(5-ethylpyrimidin-2-yl)piperidin-4-yl)thiazole (M1BX2982) or 3-isopropyl-5-(4-(((6-(4-(methylsulfonyl)phenyl)pyridin-3-yl)oxy)methyl)piperidin-1-yl)-1,2,4-oxadiazole (GSK1292263).
